SEQUENCE LISTING (8 sequences)

SEQ ID NO: 1 - DNA, 21 bp, Photinus pyralis
cuuacgcugaguacuucgatt

SEQ ID NO: 2 - DNA, 21 bp, Photinus pyralis
ucgaaguacucagcguaagtt

SEQ ID NO: 3 - DNA, 21 bp, Escherichia coli
gcgauaagucgugucuuactt

SEQ ID NO: 4 - DNA, 21 bp, Escherichia coli
guaagacacgacuuaucgctt

SEQ ID NO: 5 - DNA, 21 bp, Homo sapiens
agggcaacuuccagaccautt

SEQ ID NO: 6 - DNA, 21 bp, Homo sapiens
auggucuggaaguugcccutt

SEQ ID NO: 7 - DNA, 21 bp, Mus musculus
cacucagucucuaagggcutt

SEQ ID NO: 8 - DNA, 21 bp, Mus musculus
agcccuuagagacugagugtt

(19) United States
(12) Patent Application Publication
(10) Pub. No.: US 2005/0260270 A1
(43) Pub. Date: Nov. 24, 2005

(54) Inhibition of gene expression by delivery of small interfering RNA to post-embryonic animal cells in vivo

(76) Inventors: David L. Lewis, Madison, WI (US); Hans Herweijer, Madison, WI (US); Jon A. Wolff, Madison, WI (US); James E. Hagstrom, Middleton, WI (US); Aaron G. Loomis, Prairie du Sac, WI (US)

Correspondence Address: MIRUS CORPORATION, 505 SOUTH ROSA RD, MADISON, WI, 53719, US

(21) Appl. No.: 11/113,586

(22) Filed: Apr. 25, 2005

Related U.S. Application Data
(63) Continuation-in-part of application No. 10/007,459, filed on Nov. 7, 2001.
Application No. 10/007,459 is a continuation-in-part of application No. 09/450,315, filed on Nov. 29, 1999, now Pat. No. 6,379,966.
(60) Provisional application No. 60/315,394, filed on Aug. 27, 2001.
(60) Provisional application No. 60/324,155, filed on Sep. 20, 2001.

(51) Int. Cl. A61K 48/00; A61K 9/14

(52) U.S. Cl. 424/486; 514/44

(57) ABSTRACT

A process is provided to deliver small interfering RNA to cells in vivo for the purpose of inhibiting gene expression in that cell. The small interfering RNA is less than 50 base-pairs in length. This process is practiced on post-embryonic animals. Inhibition is sequence-specific and relies on sequence identity of the small interfering RNA and the target nucleic acid molecule.

This application is divisional of application Ser. No. 10/007,459, filed Nov. 7, 2001, which is a continuation-in-part of application Ser. No. 09/450,315, filed Nov. 29, 1999, and claims the benefit of U.S. Provisional Application Nos. 60/315,394 filed Aug. 27, 2001, and 60/324,155 filed Sep. 20, 2001. Application Ser. Nos. 10/007,459 and 09/450,315 are incorporated herein by reference.

### FIELD

The present invention generally relates to inhibiting gene expression. Specifically, it relates to inhibiting gene expression by delivery of small interfering RNAs (siRNAs) to post-embryonic animals.

### BACKGROUND

RNA interference (RNAi) describes the phenomenon whereby the presence of double-stranded RNA (dsRNA) of sequence that is identical or highly similar to a target gene results in the degradation of messenger RNA (mRNA) transcribed from that targeted gene (Sharp 2001). RNAi is likely mediated by siRNAs of approximately 21-25 nucleotides in length which are generated from the input dsRNAs (Hammond, Bernstein et al. 2000; Parrish, Fleenor et al. 2000; Yang, Lu et al. 2000; Zamore, Tuschl et al. 2000; Bernstein, Caudy et al. 2001).

The ability to specifically knock-down expression of a target gene by RNAi has obvious benefits. For example, RNAi could be used to generate animals that mimic true genetic “knockout” animals to study gene function. In addition, many diseases arise from the abnormal expression of a particular gene or group of genes. RNAi could be used to inhibit the expression of the genes and therefore alleviate symptoms of or cure the disease. For example, genes contributing to a cancerous state could be inhibited. In addition, viral genes could be inhibited, as well as mutant genes causing dominant genetic diseases such as myotonic dystrophy. Inhibiting such genes as cyclooxygenase or cytokines could also treat inflammatory diseases such as arthritis. Nervous system disorders could also be treated. Examples of targeted organs would include the liver, pancreas, spleen, skin, brain, prostrate, heart etc.

The introduction of dsRNA into mammalian cells is known to induce an interferon response which leads to a general block in protein synthesis and leads to cell both by both nonapoptotic and apoptotic pathways (Clemens and Elia 1997). In fact, studies performed using mammalian cells in culture indicate that introduction of long, double-stranded RNA does not lead to specific inhibition of expression of the target gene (Tuschl, Zamore et al. 1999; Caplen, Fleenor et al. 2000). A major component of the interferon response is the dsRNA-dependent protein kinase, PKR that phosphorylates and inactivates the elongation factor eIF2a. In addition, dsRNA induces the synthesis of 2′-5′ polyadenylic acid leading to the activation of the non-sequence specific RNase, RNaseL) (Player and Torrence 1998). PKR is not activated by dsRNA of less than 30 base pairs in length (Minks, West et al. 1979; Manche, Green et al. 1992).

In mammals, it has previously been demonstrated that long double-stranded RNA can be used to inhibit target gene expression in mouse oocytes and embryos (Svoboda, Stein et al. 2000; Wianny and Zernicka-Goetz 2000). It is likely that the interferon response pathway is not present in these cells at this early developmental stage. Recently, it has been shown that siRNA<30 bp can be used to induce RNAi in mammalian cells in culture (Caplen, Parrish et al. 2001; Elbashir, Harborth et al. 2001). These siRNAs do not appear to induce the interferon response in mammalian cells in culture. One reason for this may be that these siRNAs are too small to activate PKR.

Researchers have always been pessimistic about applying RNAi to mammalian cells because exposing such cells to dsRNA, of any sequence, triggers a global shut down of protein synthesis. Additionally, the process of effectively delivering siRNAs to mammalian cells in an animal (noninvasive transportation of the siRNA to the cell) will be difficult. (Nature, v. 411, p. 428-429, May, 2001)

### SUMMARY

We describe, in a preferred embodiment, a complex for inhibiting nucleic acid expression in a cell. The complex comprises mixing a siRNA and a compound to form the complex wherein the zeta potential of the complex is less negative than the zeta potential of the siRNA alone. Then inserting the complex into a mammalian blood vessel, in vivo, and delivering the complex to the cell wherein the nucleic acid expression is inhibited.

In another preferred embodiment, we describe a process for delivering the complex of claim 1 into a cell of a mammal. The process comprises making the siRNA-compound complex wherein the compound is selected from the group consisting of amphipathic compounds, polymers and non-viral vectors. Then the complex is inserted into a mammalian vessel and the vessel fluid delivers the siRNA to the cell.

### DETAILED DESCRIPTION

We have found that an intravascular route of administration allows a polynucleotide to be delivered to a parenchymal cell in a more even distribution than direct parenchymal injections. The efficiency of polynucleotide delivery and expression may be increased by increasing the permeability of the tissue's blood vessel. Permeability is increased by increasing the intravascular hydrostatic (physical) pressure, delivering the injection fluid rapidly (injecting the injection fluid rapidly), using a large injection volume, and increasing permeability of the vessel wall.

The term intravascular refers to an intravascular route of administration that enables a polynucleotide to be delivered to cells. Intravascular herein means within an internal tubular structure called a vessel that is connected to a tissue or organ within the body of an animal, including mammals. Within the cavity of the tubular structure, a bodily fluid flows to or from the body part. Examples of bodily fluid include blood, lymphatic fluid, or bile. Examples of vessels include arteries, arterioles, capillaries, venules, sinusoids, veins, lymphatics, and bile ducts. The intravascular route includes delivery through the blood vessels such as an artery or a vein.

Afferent blood vessels of organs are defined as vessels in which blood flows toward the organ or tissue under normal physiologic conditions. Efferent blood vessels are defined as vessels in which blood flows away from the organ or tissue under normal physiologic conditions. In the heart, afferent vessels are known as coronary arteries, while efferent vessels are referred to as coronary veins.

Volume means the amount of space that is enclosed within an object or solid shape such as an organ.

Zeta potential is the difference in electrical potential between a tightly bound layer of ions on particle surfaces and the liquid in which the particles are suspended.

Parenchymal cells are the distinguishing cells of a gland or organ contained in and supported by the connective tissue framework. The parenchymal cells typically perform a function that is unique to the particular organ. The term “parenchymal” often excludes cells that are common to many organs and tissues such as fibroblasts and endothelial cells within blood vessels.

In a liver organ, the parenchymal cells include hepatocytes, Kupffer cells and the epithelial cells that line the biliary tract and bile ductules. The major constituent of the liver parenchyma are polyhedral hepatocytes (also known as hepatic cells) that presents at least one side to an hepatic sinusoid and opposed sides to a bile canaliculus. Liver cells that are not parenchymal cells include cells within the blood vessels such as the endothelial cells or fibroblast cells. In one preferred embodiment hepatocytes are targeted by injecting the polynucleotide within the tail vein of a rodent such as a mouse.

In striated muscle, the parenchymal cells include myoblasts, satellite cells, myotubules, and myofibers. In cardiac muscle, the parenchymal cells include the myocardium also known as cardiac muscle fibers or cardiac muscle cells and the cells of the impulse connecting system such as those that constitute the sinoatrial node, atrioventricular node, and atrioventricular bundle.

The term nucleic acid is a term of art that refers to a string of at least two base-sugar-phosphate combinations. For naked DNA delivery, a polynucleotide contains more than 120 monomeric units since it must be distinguished from an oligonucleotide. However, for purposes of delivering RNA, RNAi and siRNA, either single or double stranded, a polynucleotide contains 2 or more monomeric units. Nucleotides are the monomeric units of nucleic acid polymers. The term includes deoxyribonucleic acid (DNA) and ribonucleic acid (RNA) in the form of a messenger RNA, anti-sense, plasmid DNA, parts of a plasmid DNA or genetic material derived from a virus. Anti-sense is a polynucleotide that interferes with the function of DNA and/or RNA. The term nucleic acids—refers to a string of at least two base-sugar-phosphate combinations. Natural nucleic acids have a phosphate backbone, artificial nucleic acids may contain other types of backbones, but contain the same bases. Nucleotides are the monomeric units of nucleic acid polymers. The term includes deoxyribonucleic acid (DNA) and ribonucleic acid (RNA). RNA may be in the form of an tRNA (transfer RNA), snRNA (small nuclear RNA), rRNA (ribosomal RNA), mRNA (messenger RNA), anti-sense RNA, RNAi, siRNA, and ribozymes. The term also includes PNAs (peptide nucleic acids), phosphorothioates, and other variants of the phosphate backbone of native nucleic acids.

Double-stranded RNA that is responsible for inducing RNAi is termed interfering RNA. The term siRNA means short interfering RNA which is double-stranded RNA that is less than 30 bases and preferably 21-25 bases in length.

A polynucleotide can be delivered to a cell to express an exogenous nucleotide sequence, to inhibit, eliminate, augment, or alter expression of an endogenous nucleotide sequence, or to express a specific physiological characteristic not naturally associated with the cell. Polynucleotides may be anti-sense.

We demonstrate that delivery of siRNA to cells of post-embryonic mice and rats interferes with specific gene expression in those cells. The inhibition is gene specific and does not cause general translational arrest. Thus RNAi can be effective in post-embryonic mammalian cells in vivo.

In another preferred embodiment, the permeability of the vessel is increased. Efficiency of polynucleotide delivery and expression was increased by increasing the permeability of a blood vessel within the target tissue. Permeability is defined here as the propensity for macromolecules such as polynucleotides to move through vessel walls and enter the extravascular space. One measure of permeability is the rate at which macromolecules move through the vessel wall and out of the vessel. Another measure of permeability is the lack of force that resists the movement of polynucleotides being delivered to leave the intravascular space.

To obstruct, in this specification, is to block or inhibit inflow or outflow of blood in a vessel. Rapid injection may be combined with obstructing the outflow to increase permeability. For example, an afferent vessel supplying an organ is rapidly injected and the efferent vessel draining the tissue is ligated transiently. The efferent vessel (also called the venous outflow or tract) draining outflow from the tissue is also partially or totally clamped for a period of time sufficient to allow delivery of a polynucleotide. In the reverse, an efferent is injected and an afferent vessel is occluded.

In another preferred embodiment, the intravascular pressure of a blood vessel is increased by increasing the osmotic pressure within the blood vessel. Typically, hypertonic solutions containing salts such as NaCl, sugars or polyols such as mannitol are used. Hypertonic means that the osmolarity of the injection solution is greater than physiologic osmolarity. Isotonic means that the osmolarity of the injection solution is the same as the physiological osmolarity (the tonicity or osmotic pressure of the solution is similar to that of blood). Hypertonic solutions have increased tonicity and osmotic pressure similar to the osmotic pressure of blood and cause cells to shrink.

In another preferred embodiment, the permeability of the blood vessel can also be increased by a biologically-active molecule. A biologically-active molecule is a protein or a simple chemical such as papaverine or histamine that increases the permeability of the vessel by causing a change in function, activity, or shape of cells within the vessel wall such as the endothelial or smooth muscle cells. Typically, biologically-active molecules interact with a specific receptor or enzyme or protein within the vascular cell to change the vessel's permeability. Biologically-active molecules include vascular permeability factor (VPF) which is also known as vascular endothelial growth factor (VEGF). Another type of biologically-active molecule can also increase permeability by changing the extracellular connective material. For example, an enzyme could digest the extracellular material and increase the number and size of the holes of the connective material.

In another embodiment a non-viral vector along with a polynucleotide is intravascularly injected in a large injection volume. The injection volume is dependent on the size of the animal to be injected and can be from 1.0 to 3.0 ml or greater for small animals (i.e. tail vein injections into mice). The injection volume for rats can be from 6 to 35 ml or greater. The injection volume for primates can be 70 to 200 ml or greater. The injection volumes in terms of ml/body weight can be 0.03 ml/g to 0.1 ml/g or greater.

The injection volume can also be related to the target tissue. For example, delivery of a non-viral vector with a polynucleotide to a limb can be aided by injecting a volume greater than 5 ml per rat limb or greater than 70 ml for a primate. The injection volumes in terms of ml/limb muscle are usually within the range of 0.6 to 1.8 ml/g of muscle but can be greater. In another example, delivery of a polynucleotide to liver in mice can be aided by injecting the non-viral vector-polynucleotide in an injection volume from 0.6 to 1.8 ml/g of liver or greater. In another preferred embodiment, delivering a polynucleotide-non-viral vector to a limb of a primate (rhesus monkey), the complex can be in an injection volume from 0.6 to 1.8 ml/g of limb muscle or anywhere within this range.

In another embodiment the injection fluid is injected into a vessel rapidly. The speed of the injection is partially dependent on the volume to be injected, the size of the vessel to be injected into, and the size of the animal. In one embodiment the total injection volume (1-3 mls) can be injected from 15 to 5 seconds into the vascular system of mice. In another embodiment the total injection volume (6-35 mls) can be injected into the vascular system of rats from 20 to 7 seconds. In another embodiment the total injection volume (80-200 mls) can be injected into the vascular system of monkeys from 120 seconds or less.

In another embodiment a large injection volume is used and the rate of injection is varied. Injection rates of less than 0.012 ml per gram (animal weight) per second are used in this embodiment. In another embodiment injection rates of less than ml per gram (target tissue weight) per second are used for gene delivery to target organs. In another embodiment injection rates of less than 0.06 ml per gram (target tissue weight) per second are used for gene delivery into limb muscle and other muscles of primates.

There are three types of reporter (marker) gene products that are expressed from reporter genes. The reporter gene/protein systems include:
a) Intracellular gene products such as luciferase, β-galactosidase, or chloramphenicol acetyl transferase. Typically, they are enzymes whose enzymatic activity can be easily measured.
b) Intracellular gene products such as β-galactosidase or green fluorescent protein which identify cells expressing the reporter gene. On the basis of the intensity of cellular staining, these reporter gene products also yield qualitative information concerning the amount of foreign protein produced per cell.
c) Secreted gene products such as secreted alkaline phosphatase (SEAP), growth hormone, factor IX, or alpha1-antitrypsin are useful for determining the amount of a secreted protein that a gene transfer procedure can produce. The reporter gene product can be assayed in a small amount of blood.

In a preferred embodiment, we provide a process for inhibiting gene expression in post-embryonic mammalian cells in vivo by delivering to a mammalian cell a siRNA comprising a double-stranded structure having a nucleotide sequence substantially identical to a sequence contained within the target gene and verifying the inhibition of expression of the target gene.

We also provide a process for delivery of siRNA to the cells of post-embryonic mammals. Specifically, this method is pressurized intravascular injection of siRNA, which are delivered to cells in vivo.

Additionally, another preferred embodiment provides a process for the delivery of siRNA to the cells of post-embryonic mammals. Specifically, this method is delivery of nucleic acids to cells via bile duct injection.

Yet another preferred embodiment provides for delivery of siRNA to the cells of post-embryonic mammals to muscle cells via pressurized injection of the iliac artery.

### EXAMPLES

The following examples are provided in order to demonstrate and further illustrate certain preferred embodiments and aspects of the present invention and are not to be construed as limiting the scope thereof.

### Example 1

### Inhibition of Luciferase Gene Expression by siRNA in Liver Cells In Vivo

A. Preparation of siRNA—Single-stranded, gene-specific sense and antisense RNA oligomers with overhanging 3′ deoxynucleotides are prepared and purified by PAGE. The two oligomers, 40 μM each, are annealed in 250 μl of buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA is stored at −20° C. prior to use.

The sense oligomer with identity to the luc+ gene has the sequence: 5′-rCrUrUrArCrGrCrUrGrArGrUrArCrUrUrCrGrATT-3′ (SEQ ID 1) and corresponds to positions 155-173 of the luc+ reading frame. The letter “r” preceding a nucleotide indicates that nucleotide is a ribonucleotide.

The antisense oligomer with identity to the luc+ gene has the sequence: 5′-rUrCrGrArArGrUrArCrUrCrArGrCrGrUrArArGTT-3′ (SEQ ID 2) and corresponds to positions 155-173 of the luc+ reading frame in the antisense direction. The letter “r” preceding a nucleotide indicates that nucleotide is a ribonucleotide.

The annealed oligomers containing luc+ coding sequence are referred to as siRNA-luc+.

The sense oligomer with identity to the ColE1 replication origin of bacterial plasmids has the sequence:

| | |
|---|---|
| 5′-rGrCrGrArUrArArGrUrCrGrUrGrUrCrUrUrArCTT-3′ | (SEQ ID 3) |

The letter “r” preceding a nucleotide indicates that nucleotide is a ribonucleotide.

The antisense oligomer with identity to the ColE1 origin of bacterial plasmids has the sequence:

| | |
|---|---|
| 5′-rGrUrArArGrArCrArCrGrArCrUrUrArUrCrGrCTT-3′ | (SEQ ID 4) |

The letter “r” preceding a nucleotide indicates that nucleotide is a ribonucleotide.

The annealed oligomers containing ColE1 sequence are referred to as siRNA-ori.

B. Delivery of target DNA and siRNA to liver cells in mice—Plasmid pMIR48 (10 μg), containing the luc+ coding region (Promega Corp.) and a chimeric intron downstream of the cytomegalovirus major immediate-early enhancer/promoter, is mixed with 0.5 or 5 μg of siRNA-luc+ and diluted in 1-3 mls Ringer's solution (147 mM NaCl, 4 mM KCl, 1.13 mM CaCl₂) and injected in the tail vein over 7-120 seconds.

C. Assay of Luc+ activity and assessment of siRNA induction of RNAi—One day after injection, the livers are harvested and homogenized in lysis buffer (0.1% Triton X-100, 0.1 M K-phosphate, 1 mM DTT, pH 7.8). Insoluble material is cleared by centrifugation. 10 μl of the cellular extract or extract diluted 10× is analyzed for luciferase activity using the Enhanced Luciferase Assay kit (Mirus).

Co-injection of 10 μg of pMIR48 and 0.5 μg of siRNA-luc+ results in 69% inhibition of Luc+ activity as compared to injection of 10 μg of pMIR48 alone. Co-injection of 5 μg of siRNA-luc+ with 10 μg of pMIR48 results in 93% inhibition of Luc+ activity.

### Example 2

### Inhibition of Luciferase Expression by siRNA is Gene Specific in Liver In Vivo

In this example, two plasmids are injected simultaneously with or without siRNA-luc+ as described in Example 1. The first, pMIR116, contains the luc+ coding region OIC intron under transcriptional control of the simian virus 40 enhancer and early promoter region. The second, pMIR122, contains the coding region for the Renilla reniformis luciferase under transcriptional control of the Simion virus 40 enhancer and early promoter region.

10 μg of pMIR116 and 1 μg of pMIR122 is injected as described in Example 1 without siRNA, or 0.5 or 5.0 μg siRNA-luc+. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the 0 μg siRNA-Luc+ control. siRNA-luc+ specifically inhibited the target Luc+ expression 73% at 0.5 μg co-injected siRNA-luc+ and 82% at 5.0 μg co-injected siRNA-luc+.

### Example 3

### Inhibition of Luciferase Expression by siRNA is Gene Specific and siRNA Specific in Liver In Vivo

In this Example, 10 μg of pMIR116 and 1 μg of pMIR122 is injected as described in Example 1 with 5.0 μg siRNA-luc+ or 5.0 μg siRNA-ori. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in liver by 93% compared to siRNA-ori indicating inhibition by siRNAs is sequence specific in this organ.

### Example 4

### Inhibition of Luciferase Expression by siRNA is Gene Specific and siRNA Specific in Spleen In Vivo

In this Example, 10 μg of pMIR116 and 1 μg of pMIR122 is injected as described in Example 1 with 5.0 μg siRNA-luc+ or 5.0 μg siRNA-ori. One day after injection, the spleens were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in spleen by 90% compared to siRNA-ori indicating inhibition by siRNAs is sequence specific in this organ.

### Example 5

### Inhibition of Luciferase Expression by siRNA is Gene Specific and siRNA Specific in Lung In Vivo

In this Example, 10 μg of pMIR116 and 1 μg of pMIR122 is injected as described in Example 1 with 5.0 μg siRNA-luc+ or 5.0 μg siRNA-ori. One day after injection, the lungs were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in lung by 89% compared to siRNA-ori indicating inhibition by siRNAs is sequence specific in this organ.

### Example 6

### Inhibition of Luciferase Expression by siRNA is Gene Specific and siRNAi Specific in Heart In Vivo

In this Example, 10 μg of pMIR116 and 1 μg of pMIR122 is injected as described in Example 1 with 5.0 μg siRNA-luc+ or 5.0 μg siRNA-ori. One day after injection, the hearts were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in heart by 80%.

### Example 7

### Inhibition of Luciferase Expression by siRNA is Gene Specific and siRNA Specific in Kidney In Vivo

In this Example, 10 μg of pMIR116 and 1 μg of pMIR122 is injected as described in Example 1 with 5.0 μg siRNA-luc+ or 5.0 μg siRNA-ori. One day after injection, the kidneys were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in kidney by 90% compared to siRNA-ori indicating inhibition by siRNAs is sequence specific in this organ.

### Example 8

### Inhibition of Luciferase Expression by siRNA is Gene Specific and siRNA Specific in Liver after Bile Duct Delivery In Vivo

In this example, 10 μg of pMIR116 and 1 μg of pMIR122 with 5.0 μg siRNA-luc+ or 5.0 μg siRNA-ori are injected into the bile duct of mice in a total volume of 1 ml in Ringer's buffer delivered at 6 ml/min. The inferior vena cava is clamped above and below the liver before injection are left on for two minutes after injection. One day after injection, the liver is harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in kidney by 88% compared to the control siRNA-ori.

### Example 9

### Inhibition of Luciferase Expression by siRNA is Gene Specific and siRNA Specific in Muscle In Vivo after Intravascular Delivery

In this example, 10 μg of pMIR116 and 1 μg of pMIR122 with 5.0 μg siRNA-luc+ or 5.0 μg siRNA-ori were injected into iliac artery of rats under high pressure. Specifically, animals are anesthetized and the surgical field shaved and prepped with an antiseptic. The animals are placed on a heating pad to prevent the loss of body heat during the surgical procedure. A midline abdominal incision will be made after which skin flaps will be folded away with clamps to expose the target area. A moist gauze will be applied to prevent excessive drying of internal organs. Intestines will be moved to visualize the iliac veins and arteries. Microvessel clips are placed on the external iliac, caudal epigastric, internal iliac, deferent duct, and gluteal arteries and veins to block both outflow and inflow of the blood to the leg. An efflux enhancer solution (e.g., 0.5 mg papaverine in 3 ml saline) is injected into the external iliac artery though a 25-27 g needle, followed by the plasmid DNA and siRNA containing solution (in 10 ml saline) 1-10 minutes later. The solution is injected in approximately 10 seconds. The microvessel clips are removed 2 minutes after the injection and bleeding controlled with pressure and gel foam. The abdominal muscles and skin are closed with 4-0 dexon suture. Each procedure takes approximately 15 minutes to perform.

Four days after injection, rats were sacrificed and the quadricep and gastrocnemius muscles were harvested and homogenized as described in Example 1. Luc+ and Renilla Luc activities were assayed using the Dual Luciferase Reporter Assay System (Promega). Ratios of Luc+ to Renilla Luc were normalized to the siRNA-ori control. siRNA-Luc+ inhibited Luc+ expression in qaudriceps and gastrocnemius by 85% and 92%, respectively, compared to the control siRNA-ori.

### Example 10

### RNAi of SEAP Reporter Gene Expression Using siRNA In Vivo

Single-stranded, SEAP-specific sense and antisense RNA oligomers with overhanging 3′ deoxynucleotides are prepared and purified by PAGE. The two oligomers, 40 μM each, are annealed in 250 μl of buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA is stored at −20° C. prior to use.

The sense oligomer with identity to the SEAP reporter gene has the sequence: 5′-rArGrGrGrCrArArCrUrUrCrCrArGrArCrCrArUTT-3′ (SEQ ID 5) and corresponds to positions 362-380 of the SEAP reading frame in the sense direction. The letter “r” preceding a nucleotide indicates that nucleotide is a ribonucleotide.

The antisense oligomer with identity to the SEAP reporter gene has the sequence: 5′-rArUrGrGrUrCrUrGrGrArArGrUrUrGrCrCrCrUTT-3′ (SEQ ID 6) and corresponds to positions 362-380 of the SEAP reading frame in the antisense direction. The letter “r” preceding a nucleotide indicates that nucleotide is a ribonucleotide.

The annealed oligomers containing SEAP coding sequence are referred to as siRNA-SEAP.

Plasmid pMIR141 (10 μg), containing the SEAP coding region (Promega Corp.) under transcriptional control of the human ubiquitin C promoter and the human hepatic control region of the apolipoprotein E gene cluster, is mixed with 0.5 or 5 μg of siRNA-SEAP or 5 μg siRNA-ori and diluted in 1-3 mls Ringer's solution (147 mM NaCl, 4 mM KCl, 1.13 mM CaCl₂) and injected in the tail vein over 7-120 seconds. Control mice also include those injected with pMIR141 alone.

Each mouse is bled from the retro-orbital sinus one day after injection. Cells and clotting factors are pelleted from the blood to obtain serum. The serum is evaluated for the presence of SEAP by a chemiluminescence assay using the Tropix Phospha-Light kit.

Results indicate SEAP expression was inhibited by 59% when 0.5 μg siRNA-SEAP was delivered and 83% when 5.0 μg siRNA-SEAP was delivered. No decrease in SEAP expression was observed when 5.0 μg of siRNA-ori was delivered indicating the decrease in SEAP expression by siRNA-SEAP is gene specific.

Day 1

| | AVE SEAP (ng/ml) | SD |
|---|---|---|
| plasmid only | 2239 | 1400 |
| siRNA-ori (5.0 μg) | 2897 | 1384 |
| siRNA-SEAP (0.5 μg) | 918 | 650 |
| siRNA-SEAP (5.0 μg) | 384 | 160 |

### Example 11

### Inhibition of Endogenous Mouse Cytosolic Alanine Aminotransferase (ALT) Expression after In Vivo Delivery of siRNA

Single-stranded, cytosolic alanine aminotrasferase-specific sense and antisense RNA oligomers with overhanging 3′ deoxynucleotides are prepared and purified by PAGE. The two oligomers, 40 μM each, are annealed in 250 μl of buffer containing 50 mM Tris-HCl, pH 8.0 and 100 mM NaCl, by heating to 94° C. for 2 minutes, cooling to 90° C. for 1 minute, then cooling to 20° C. at a rate of 1° C. per minute. The resulting siRNA is stored at −20° C. prior to use.

The sense oligomer with identity to the endogenous mouse and rat gene encoding cytosolic alanine aminotransferase has the sequence: 5′-rCrArCrUrCrArGrUrCrUrCrUrArArGrGrGrCrUTT-3′ (SEQ ID 7) and corresponds to positions 928-946 of the cytosolic alanine aminotransferase reading frame in the sense direction. The letter “r” preceding a nucleotide indicates that nucleotide is a ribonucleotide.

The sense oligomer with identity to the endogenous mouse and rat gene encoding cytosolic alanine aminotransferase has the sequence: 5′-rArGrCrCrCrUrUrArGrArGrArCrUrGrArGrUrGTT-3′ (SEQ ID 8) and corresponds to positions 928-946 of the cytosolic alanine aminotransferase reading frame in the antisense direction. The letter “r” preceding a nucleotide indicates that nucleotide is a ribonucleotide.

The annealed oligomers containing cytosolic alanine aminotransferase coding sequence are referred to as siRNA-ALT

Mice are injected in the tail vein over 7-120 seconds with 40 μg of siRNA-ALT diluted in 1-3 mls Ringer's solution (147 mM NaCl, 4 mM KCl, 1.13 mM CaCl₂). Control mice were injected with Ringer's solution without siRNA. Two days after injection, the livers were harvested and homogenized in 0.25 M sucrose. ALT activity was assayed using the Sigma diagnostics INFINITY ALT reagent according to the manufacturers instructions. Total protein was determined using the BioRad Protein Assay. Mice injected with 40 μg of siRNA-ALT had a 32% average decrease in ALT specific activity compared to that of mice injected with Ringer's solution alone.

### Example 12

### We have Achieved Expression of the LDL Receptor in Low-Density Lipoprotein Receptor (LDLR) (−/−) Mice, Which Lowers Triglycerides

For these experiments, mice lacking the LDLR were used. These mice have elevated lipoprotein levels. Expression of the LDLR in the liver is expected to result in lowering of lipoproteins. To this end, 100 μg of pCMV-LDLR was injected into the bile duct of LDLR (−/−) mice (obtained form The Jackson Laboratories). Blood was obtained one day prior and one day after plasmid DNA injection and analyzed for triglycerides levels. The average triglycerides level before injection was 209±69 mg/dl. One day after pDNA delivery, triglyceride levels were measured at 59±14 mg/dl. We included a few normal mice, in which triglyceride levels were lowered as well.

### Example 13

### Synthesis of L-cystine-1,4-bis(3-aminopropyl)piperazine copolymer (M66)

To a solution of N,N′-Bis(t-BOC)-L-cystine (85 mg, 0.15 mmol) in ethyl acetate (20 ml) was added N,N′-dicyclohexylcarbodiimide (108 mg, 0.5 mmol) and N-hyroxysuccinimide (60 mg, 0.5 mmol). After 2 hr, the solution was filtered through a cotton plug and 1,4-bis(3-aminopropyl)piperazine (54 μL, 0.25 mmol) was added. The reaction was allowed to stir at room temperature for 16 h. The ethyl acetate was then removed by rotary evaporation and the resulting solid was dissolved in trifluoroacetic acid (9.5 ml), water (0.5 ml) and triisopropylsilane (0.5 ml). After 2 h, the trifluoroacetic acid was removed by rotary evaporation and the aqueous solution was dialyzed in a 15,000 MW cutoff tubing against water (2×2 l) for 24 h. The solution was then removed from dialysis tubing, filtered through 5 μM nylon syringe filter and then dried by lyophilization to yield 30 mg of polymer.

### Example 14

### Luciferase Expression in a Variety of Tissues Following a Single Tail Vein Injection of pCILuc/66 Complexes

DNA and polymer 66 were mixed at a 1:1.7 wt:wt ratio in water and diluted to 2.5 ml with Ringers solution as described. Complexes were injected into tail vein of 25 g ICR mice within 7 seconds. Mice were sacrificed 24 hours after injection and various organs were assayed for luciferase expression.

| Organ | Total Relative Light Units |
|---|---|
| Prostate | 637,000 |
| Skin (abdominal wall) | 194,000 |
| Testis | 589,000 |
| Skeletal Muscle (quadriceps) | 35,000 |
| fat (peritoneal cavity) | 44,700 |
| bladder | 17,000 |
| brain | 247,000 |
| pancreas | 2,520,000 |

### Example 15

### Directed Intravascular Injection of pCILuc/66 Polymer Complexes into Dorsal Vein of Penis Results in High Level Gene Expression in the Prostate and Other Localized Tissues

Complexes were formed as described for example above and injected rapidly into the dorsal vein of the penis (within 7 seconds). For directed delivery to the prostate with increased hydrostatic pressure, clamps were applied to the inferior vena cava and the anastomotic veins just prior to the injection and removed just after the injection (within 5-10 seconds). Mice were sacrificed 24 hours after injection and various organs were assayed for luciferase expression.

| Organ | Total Relative Light Units per organ |
|---|---|
| Prostate | 129,982,450 |
| Testis | 4,229,000 |
| fat (around bladder) | 730,300 |
| bladder | 618,000 |

### Example 16

### Injection of Plasmid DNA (pCILuc)/L-cystine-1,4-bis(3-aminopropyl)piperazine Copolymer (M66) Complexes into the Iliac Artery of Rats

Complex formation—500 ug pDNA (500 ul) was mixed with M66 copolymer at a 1:3 wt:wt ratio in 500 ul saline. Complexes were then diluted in Ringers solution to total volume of 10 mls. Injections—total volume of 10 mls was injected into the iliac artery of Sprague-Dawley rats (Harlan, Indianapolis, Ind.) in approximately 10 seconds. Expression—Animals were sacrificed after 1 week and individual muscle groups were removed and assayed for luciferase expression.

| Rat hind limb muscle groups | Relative Light Units |
|---|---|
| upper leg posterior | 6.46 × 10⁸ total (32 ng luciferase) |
| upper leg anterior | 3.58 × 10⁹ total (183 ng luciferase) |
| upper leg middle | 2.63 × 10⁹ total (134 ng luciferase) |
| lower leg anterior | 3.19 × 10⁹ total (163 ng luciferase) |
| lower leg anterior | 1.97 × 10⁹ total (101 ng luciferase) |

These results indicate that high level gene expression in all muscle groups of the leg was facilitated by intravascular delivery of pCILuc/M66 complexes into rat iliac artery.

### Example 17

### Synthesis of 5,5′-Dithiobis(2-nitrobenzoic acid)-Tetraethylenepentamine Copolymer (#M57)

Tetraethylenepentamine (3.2 μL, 0.017 mmol, Aldrich Chemical Company) was taken up in 1.0 ml dichloromethane and HCl (1 ml, 1 M in Et₂O, Aldrich Chemical Company) was added Et₂O was added and the resulting HCl salt was collected by filtration. The salt was taken up in 1 ml DMF and 5,5′-dithiobis[succinimidyl (2-nitrobenzoate)] (10 mg, 0.017 mmol) was added. The resulting solution was heated to 80° C. and diisopropylethylamine (15 μL, 0.085 mmol, Aldrich Chemical Company) was added dropwise. After 16 hr, the solution was cooled, diluted with 3 ml H₂O, and dialyzed in 12,000-14,000 MW cutoff tubing against water (2×2 L) for 24 h. The solution was then removed from dialysis tubing and dried by lyophilization to yield 5.8 mg (62%) of 5,5′-dithiobis(2-nitrobenzoic acid)-tetraethylenepentamine copolymer.

Mouse Tail Vein Injections of pDNA (pCI Luc)/5,5′-Dithiobis(2-nitrobenzoic acid)-Tetraethylenepentamine Copolymer Complexes.

Complexes were prepared as follows:
Complex I: pDNA (pCI Luc, 200 μg) was added to 300 μL DMSO then 2.5 ml Ringers was added.
Complex II: pDNA (pCI Luc, 200 μg) was added to 300 μL DMSO then 5,5′-Dithiobis(2-nitrobenzoic acid)-Tetraethylenepentamine Copolymer (336 μg) was added followed by 2.5 ml Ringers.

High pressure (2.5 ml) tail vein injections of the complex were performed as previously described (Zhang et al. 1999). Results reported are for liver expression, and are the average of two mice. Luciferase expression was determined as previously reported (Wolff et al. 1990 “Direct gene transfer into mouse muscle in vivo,” Science 247, 1465-8.) A Lumat LB 9507 luminometer was used.

Results: High pressure injections

| | |
|---|---|
| Complex I: | 25,200,000 Relative Light Units |
| Complex II: | 21,000,000 Relative Light Units |

Results indicate that pDNA (pCI Luc)/5,5′-Dithiobis(2-nitrobenzoic acid)-tetraethylenepentamine copolymer complexes are nearly equivalent to pCI Luc DNA itself in high pressure injections. This indicates that the pDNA is being released from the complex and is accessible for transcription.

### Example 18

### Synthesis of 5,5′-Dithiobis(2-nitrobenzoic acid)-Tetraethylenepentamine-Tris(2-aminoethyl)amine Copolymer (#M58)

Tetraethylenepentamine (2.3 μL, 0.012 mmol, Aldrich Chemical Company) and tris(2-aminoethyl)amine (0.51 μL, 0.0034 mmol, Aldrich Chemical Company) were taken up in 0.5 ml methanol and HCl (1 ml, 1 M in Et₂O, Aldrich Chemical Company) was added. Et₂O was added and the resulting HCl salt was collected by filtration. The salt was taken up in 1 ml DMF and 5,5′-dithiobis[succinimidyl (2-nitrobenzoate)] (10 mg, 0.017 mmol) was added. The resulting solution was heated to 80° C. and diisopropylethylamine (15 μL, 0.085 mmol, Aldrich Chemical Company) was added dropwise. After 16 hr, the solution was cooled, diluted with 3 ml H₂O, and dialyzed in 12,000-14,000 MW cutoff tubing against water (2×2 L) for 24 h. The solution was then removed from dialysis tubing and dried by lyophilization to yield 6.9 mg (77%) of 5,5′-dithiobis(2-nitrobenzoic acid)-tetraethylenepentamine-tris(2-aminoethyl)amine copolymer.

Mouse Tail Vein Injections of pDNA (pCI Luc)/5,5′-Dithiobis(2-nitrobenzoic acid)-Tetraethylenepentamine-Tris(2-aminoethyl)amine Copolymer Complexes:

Complexes were prepared as follows:
Complex I: pDNA (pCI Luc, 200 μg) was added to 300 μL DMSO then 2.5 ml Ringers was added.
Complex II: pDNA (pCI Luc, 200 μg) was added to 300 μL DMSO then 5,5′-Dithiobis(2-nitrobenzoic acid)-Tetraethylenepentamine-Tris(2-aminoethyl)amine Copolymer (324 μg) was added followed by 2.5 ml Ringers.

High pressure (2.5 ml) tail vein injections of the complex were performed as previously described. Results reported are for liver expression, and are the average of two mice. Luciferase expression was determined a previously shown.

Results: High pressure injections

| | |
|---|---|
| Complex I: | 25,200,000 Relative Light Units |
| Complex II: | 37,200,000 Relative Light Units |

Results indicate that pDNA (pCI Luc)/5,5′-Dithiobis(2-nitrobenzoic acid)-tetraethylenepentamine-Tris(2-aminoethyl)amine Copolymer Complexes are more effective than pCI Luc DNA in high pressure injections. This indicates that the pDNA is being released from the complex and is accessible for transcription.

### Example 19

### Synthesis of guanidino-L-cystine, 1,4-bis(3-aminopropyl)piperazine Copolymer (#M67)

To a solution of cystine (1 gm, 4.2 mmol) in ammonium hydroxide (10 ml) in a screw-capped vial was added O-methylisourea hydrogen sulfate (1.8 gm, 10 mmol). The vial was sealed and heated to 60° C. for 16 h. The solution was then cooled and the ammonium hydroxide was removed by rotary evaporation. The solid was then dissolved in water (20 ml), filtered through a cotton plug. The product was then isolated by ion exchange chromatography using Bio-Rex 70 resin and eluting with hydrochloric acid (100 mM).

### Synthesis of guanidino-L-cystine1,4-bis(3-aminopropyl)piperazine Copolymer

To a solution of guanidino-L-cystine (64 mg, 0.2 mmol) in water (10 ml) was slowly added N,N′-dicyclohexylcarbodiimide (82 mg, 0.4 mmol) and N-hyroxysuccinimide (46 mg, 0.4 mmol) in dioxane (5 ml). After 16 hr, the solution was filtered through a cotton plug and 1,4-bis(3-aminopropyl)piperazine (40 μL, 0.2 mmol) was added. The reaction was allowed to stir at room temperature for 16 h and then the aqueous solution was dialyzed in a 15,000 MW cutoff tubing against water (2×2 l) for 24 h. The solution was then removed from dialysis tubing, filtered through 5 μM nylon syringe filter and then dried by lyophilization to yield 5 mg of polymer.

Particle size of pDNA-L-cystine-1,4-bis(3-aminopropyl)piperazine copolymer and DNA-guanidino-L-cystine1,4-bis(3-aminopropyl)piperazine copolymer complexes: To a solution of pDNA (10 μg/ml) in 0.5 ml 25 mM HEPES buffer pH 7.5 was added 10 μg/ml L-cystine-1,4-bis(3-aminopropyl)piperazine copolymer or guanidino-L-cystine1,4-bis(3-aminopropyl)piperazine copolymer. The size of the complexes between DNA and the polymers were measured. For both polymers, the size of the particles were approximately 60 nm.

Condensation of DNA with L-cystine-1,4-bis(3-aminopropyl)piperazine copolymer and decondensation of DNA upon addition of glutathione. Fluorescein labeled DNA was used for the determination of DNA condensation in complexes with L-cystine-1,4-bis(3-aminopropyl)piperazine copolymer. pDNA was modified to a level of 1 fluorescein per 100 bases using Mirus' LabelIT Fluorescein kit. The fluorescence was determined using a fluorescence spectrophotometer (Shimadzu RF-1501 spectrofluorometer) at an excitation wavelength of 495 nm and an emission wavelength of 530 nm (Trubetskoy, V. S., Slattum, P. M., Hagstrom, J. E., Wolff, J. A., and Budker, V. G., “Quantitative assessment of DNA condensation,” Anal Biochem 267, 309-13 (1999), incorporated herein by reference).

The intensity of the fluorescence of the fluorescein-labeled DNA (10 μg/ml) in 0.5 ml of 25 mM HEPES buffer pH 7.5 was 300 units. Upon addition of 10 μg/ml of L-cystine-1,4-bis(3-aminopropyl)piperazine copolymer, the intensity decreased to 100 units. To this DNA-polycation sample was added 1 mM glutathione and the intensity of the fluorescence was measured. An increase in intensity was measured to the level observed for the DNA sample alone. The half life of this increase in fluorescence was 8 minutes. The experiment indicates that DNA complexes with physiologically-labile disulfide-containing polymers are cleavable in the presence of the biological reductant glutathione.

Mouse Tail Vein Injection of DNA-L-cystine-1,4-bis(3-aminopropyl)piperazine copolymer and DNA-guanidino-L-cystine1,4-bis(3-aminopropyl)piperazine copolymer Complexes: Plasmid delivery in the tail vein of ICR mice was performed as previously described. To pCILuc DNA (50 μg) in 2.5 ml H₂O was added either L-cystine-1,4-bis(3-aminopropyl)piperazine copolymer, guanidino-L-cystine1,4-bis(3-aminopropyl)piperazine copolymer, or poly-L-lysine (34,000 MW, Sigma Chemical Company) (50 μg). The samples were then injected into the tail vein of mice using a 30 gauge, 0.5 inch needle. One day after injection, the animal was sacrificed, and a luciferase assay was conducted.

| Polycation | ng/liver |
|---|---|
| poly-L-lysine | 6.2 |
| L-cystine-1,4-bis(3-aminopropyl)piperazine copolymer | 439 |
| guanidino-L-cystine1,4-bis(3-aminopropyl)piperazine copolymer | 487 |

The experiment indicates that DNA complexes with the physiologically-labile disulfide-containing polymers are capable of being broken, thereby allowing the luciferase gene to be expressed.

### Example 20

### Inhibition of Luciferase Expression in Lung after In Vivo Delivery of siRNA Using Recharged Particles

Recharged particles were formed to deliver the reporter genes luciferase+ and Renilla luc as well as siRNA targeted against luciferase+ mRNA or a control siRNA to the lung. In this experiment, particles containing the reporter genes were delivered first, followed by delivery of particles containing the siRNAs. In all cases, particles were prepared with the polycation linear polyethylenimine (lPEI) and the polyanion polyacrylic acid (pAA). For delivery of reporter genes, particles were prepared which contained a mixture of the luc+ and Renilla luc expression plasmids. Normalization of expression of the two luciferase genes corrects for varying plasmid delivery efficiencies between animals. Particles containing a mixture of the expression plasmids containing the luciferase+ gene and the Renilla luciferase gene were injected intravascularly. Particles containing siRNA-Luc+ or a control siRNA were injected intravascularly immediately following injection of the plasmid-containing particles. 24 hours later, the lungs were harvested and the homogenate assayed for both Luc+ and Renilla Luc activity.

Specific experimental details were as follows: plasmid-containing particles were prepared by mixing 45 μg pGL3 control (Luc+) and 5 μg pRL-SV40 (Renilla Luc) with 300 μg lPEI in 10 mM HEPES, pH 7.5/5% glucose. After vortexing for 30 seconds, 50 μg pAA was added and the solution vortexed was for 30 seconds. siRNA-containing particles were prepared similarly, except 25 μg siRNA was used with 200 μg lPEI and 25 μg pAA. Particles containing the plasmid DNAs (total volume 250 μl) were injected into the tail vein of ICR mice. In animals that received siRNA, particles containing siRNA (total volume 100 μl) were injected into the tail vein immediately after injection of the plasmid DNA-containing particles. 1.5 mg pAA in 100 μl was then injected into the tail vein some animal 0.5 h later. 24 h later, animals were sacrificed and the lungs were harvested and homogenized. The homogenate was assayed for Luc+ and Renilla Luc activity using the Dual Luciferase Assay Kit (Promega Corporation).

Results indicate that intravascular injection of particles containing the plasmids pGL3 control and pRL-SV40 results in Luc+ and Renilla Luc expression in lung tissue (Table 2). Injection of particles containing siRNA-Luc+ after injection of the plasmid-containing particles resulted in specific inhibition of Luc+ expression. Renilla Luc expression was not inhibited. Injection of particles containing control siRNA (siRNA-c), targeted against an unrelated gene product did not result in inhibition of either Luc+ or Renilla Luc activity, demonstrating that the effect of siRNA-Luc+ on Luc+ expression is sequence specific and that injection of siRNA particles per se does not generally inhibit delivery or expression of delivered plasmid genes. These results demonstrate that particles formed with lPEI and pAA containing siRNA are able to deliver siRNA to the lung and that the siRNA cargo is biologically active once inside lung cells.

TABLE 5
Delivery of siRNA to the lung using recharged particles results in
inhibition of target gene expression.

| | Relative light units | | Average Luc+/ | Normalized |
|---|---|---|---|---|
| | Replicate | Replicate | Renilla Luc | Luc+/ |
| Particles | 1 | 2 | ratio | Renilla Luc |
| plasmids only | | | | |
| Luc+ | 560994 | 680038 | 0.43 +/− 0.05 | 1.00 |
| Renilla Luc | 1406188 | 1452593 | | |
| siRNA-Luc+ | | | | |
| Luc+ | 326697 | 428079 | 0.21 +/− 0.07 | 0.48 +/− 0.16 |
| Renilla Luc | 1283313 | 2683842 | | |
| siRNA-c | | | | |
| Luc+ | 964503 | 1452962 | 0.37 +/− 0.01 | 0.86 +/− 0.03 |
| Renilla Luc | 2527933 | 4005381 | | |

### Example 21

### In Vivo Delivery of siRNA to Mouse Liver Cells Using TransIT™In Vivo

10 μg pGL3 control and 1 μg pRL-SV40 were complexed with 11 μl TransIT™ In Vivo in 2.5 ml total volume according the manufacturer's recommendation (Mirus Corporation, Madison, Wis.). For siRNA delivery, 10 μg pGL3 control, 1 μg pRL-SV40, and either 5 μg siRNA-Luc+ or 5 μg control siRNA were complexed with 16 μl TransIT™ In Vivo in 2.5 ml total volume. Particles were injected over ˜7 s into the tail vein of 25-30 g ICR mice as described in Example 1. One day after injection, the livers were harvested and homogenized as described in Example 1. Luc+ and Renilla Luc activities were assayed using the Dual Luciferase Reporter Assay System (Promega). Ratios of Luc+ to Renilla Luc were normalized to the no siRNA control. siRNA-luc+ specifically inhibited the target Luc+ expression 96% (Table 6).

Delivery of siRNA to the Mouse Liver Using TransIT™ In Vivo Results in Inhibition of Target Gene Expression.

| | | | relative | % inhibition of |
|---|---|---|---|---|
| | | expression | LUC+ | Luc+ |
| complex | gene | (RLUs) | expression | expression |
| Plasmid alone | Luciferase | 31973057 | 5.1855 | 0.0 |
| | Renilla | 6165839 | | |
| Plasmid + siRNA- | Luciferase | 853332 | 0.2069 | 96.0 |
| Luc+ | Renilla | 4124726 | | |
| Plasmid + control | Luciferase | 5152933 | 2.1987 | 57.5 |
| SiRNA | Renilla | 2343673 | | |

These data show that the TransIT™ In Vivo labile polymer transfection reagent effectively delivers siRNA in vivo.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

1. A complex for delivering siRNA to a cell formed by the process comprising: mixing the siRNA with a compound to form the complex wherein the zeta potential of the complex is less negative than the zeta potential of the siRNA alone.

2. The complex of claim 1 wherein the complex has a negative charge.

3. The complex of claim 1 wherein the compound consists of an amphipathic compound

4. The complex of claim 1 wherein the compound consists of a polymer.

5. The complex of claim 1 wherein the compound consists of a cleavable compound.

6. The complex of claim 5 wherein the cleavable compound consists of a pH cleavable compound.

7. The complex of claim 1 wherein the compound is crosslinked to itself.

8. The complex of claim 1 wherein the complex has a positive charge.

9. The complex of claim 7 further comprising: adding a polyanion to the complex to form a ternary complex wherein the zeta potential of the ternary complex is more negative than the complex.

10. The complex of claim 8 wherein the compound is crosslinked to the polyanion.

11. The complex of claim 8 wherein the polyanion consists of a cleavable polyanion
